(19) United States
(12) Patent Application Publication
(10) Pub. No.: US 2005/0154264 A1
(43) Pub. Date: Jul. 14, 2005

(54) Personal stress level monitor and systems and methods for using same

(75) Inventors: Kellie Michelle Lecompte, Austin, TX (US); Ivan Matthew Milman, Austin, TX (US); Rahul Mishra, Austin, TX (US); Karthikeyan Ramamoorthy, Austin, TX (US)

Correspondence Address: Kelly K. Kordzik, P.O. Box 50784, 400 North Ervay Street, Dallas, TX, 75201, US

(73) Assignee: International Business Machines Corporation, Armonk, NY (US)

(21) Appl. No.: 10/753,853

(22) Filed: Jan. 8, 2004

(51) Int. Cl. A61B 5/00; A61B 10/00; G06F 17/00

(52) U.S. Cl. 600/300; 128/920; 128/903

(57) ABSTRACT

A mechanism to monitor an individual's level of stress in his or her home or workplace is provided. Unobtrusive physiologic stress senses are used in combination with a wireless link and a personal computer or other intelligent device to monitor the user's stress level. Based on a user profile and the user's baseline stress indicators, one or more stress-reducing activities are presented to the user. Additionally, if a user is in a stress-sensitive population, for example, persons with a pre-existing hypertension, the user may selectively enable additional alerts.

### TECHNICAL FIELD

The present invention relates to data processing systems and in particular to a data processing system for monitoring personal stress levels and providing remedial feedback to the user in response to elevated levels of stress.

### BACKGROUND INFORMATION

Stress is one of the major underlying causes of health problems in modern societies. Stress inducing events are common in the workplace and increasingly in events associated with the complexities of life in modern and increasingly global societies. In particular, as modern economies have evolved from industrial occupations to office jobs, productivity demands have forced workers to confront new “occupational hazards” with which they are unfamiliar and uncomfortable, personal computers being a prime example. Dealing with increasingly complex software, crashes, viruses etc. are disruptive and stressful. Consequently, managing stress is an important component in maintaining a person's health.

Stress manifests itself in different forms. Typically, physical indicia appear before the individual becomes mentally aware of his or her stressed states. For example, an increased level of stress may induce an increase in pulse rate, increased blood pressure and a change in skin conductance, among other reactions. Thus, there is a need in the art for systems and methods to use physiologic stress indicators to assist individuals in managing stress. Such a system should be usable in an unobtrusive manner in the user's local environment, such as the user's workplace, and should be secure. In particular, there is a need for a system that integrates with the user's stress-inducing activities, the personal computer or computer workstation being exemplary. Additionally, these systems and methods should provide stress management actions based on stress level indicia relative to a user's baseline.

### SUMMARY OF THE INVENTION

The aforementioned needs are addressed by the present invention. Accordingly, there is provided a method for personal stress monitoring. The method includes receiving one or more physiologic indicators. Values of the physiologic indicators are compared to corresponding baseline values. It is determined if one or more physiologic indicators or a combination of physiological indicators equals or exceeds at least one preselected threshold condition relative to the baseline values. If at least one threshold condition is equaled or exceeded, a remedial action is emitted. The remedial action corresponds to a highest level threshold condition equaled or exceeded.

The foregoing has outlined rather broadly the features and technical advantages of one or more embodiments of the present invention in order that the detailed description of the invention that follows may be better understood. Additional features and advantages of the invention will be described hereinafter which form the subject of the claims of the invention.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates, in high level block diagram form, a stress monitoring system in accordance with an embodiment of the present invention;

FIG. 2 illustrates, in block diagram form, the architecture of a stress monitoring application for use in conjunction with the system of FIG. 1;

FIGS. 3A-3B, illustrate, in flowchart form, a stress monitoring methodology in accordance with an embodiment of the present invention;

FIG. 4 illustrates, in tabular form, exemplary stress level thresholds and corresponding actions which may be used in conjunction with the methodology of FIGS. 3A-3B; and

FIG. 5 illustrates, in block diagram form, a data processing system which may be used in conjunction with the methodologies embodying the present inventive principles.

### DETAILED DESCRIPTION

A mechanism to monitor an individual's level of stress in his or her home or workplace is provided. Unobtrusive physiologic stress sensors are used in combination with a wireless link and a personal computer or other intelligent device to monitor the user's stress level. Based on a user profile and the user's baseline stress indicators, one or more stress-reducing activities are presented to the user. Additionally, if a user is in a stress-sensitive population, for example, persons with a pre-existing cardiovascular condition, the user may selectively enable additional alerts.

In the following description, numerous specific details are set forth to provide a thorough understanding of the present invention. For example, particular wireless networking protocols may be referred to, particular physiologic stress indicators or particular timing intervals may be used to illustrate the present inventive principles. However, it would be recognized by those of ordinary skill in the art that the present invention may be practiced without such specific details, and in other instances, well-known circuits have been shown in block diagram form in order not to obscure the present invention in unnecessary detail. Refer now to the drawings, wherein depicted elements are not necessarily shown to scale and wherein like or similar elements are designated by the same reference numeral through the several views.

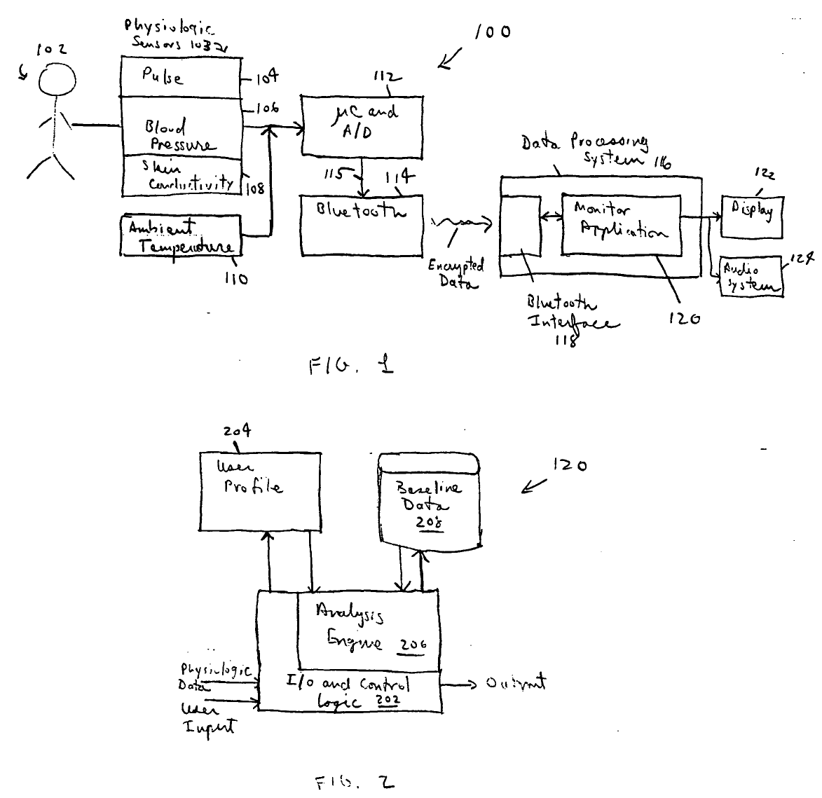

Refer now to FIG. 1 illustrating a stress monitoring system 100 in accordance with the principles of the present invention. A user 102 whose stress levels are to be monitored wears one or more physiologic sensors 103 which measure physiologic indicators commonly associated with elevated levels of stress. These may include a pulse sensor 104, a blood pressure sensor 106 or a skin conductivity sensor 108. For example, a skin conductivity sensor that may be adapted for use with the present stress monitoring system is the Galvactivator, a glove-like device that monitors the skin conductivity response due to the eccrine sweat glands of the palm of the hand, developed by the MIT Media Laboratory, Massachusetts Institute Of Technology, Cambridge, Mass. (Although the Galvactivator prototype indicates an increase in skin conductivity by illuminating an LED, it would be appreciated that the signal switching the LED may, alternatively, be used as an input to an analog-to-digital converter as discussed below. Alternatively, a digital logic level may be output when the skin conductivity exceeds a preselected threshold.) Combination of such sensors may also be used. Additionally, an ambient temperature sensor 110 may also be used in conjunction with the physiologic sensors. The ambient temperature may affect the interpretation of certain physiological indicators, such as skin conductivity, and thus, it may be desirable to include a signal representative of the ambient temperature with the physiologic sensor data. Integrated circuit temperature sensors whose output is linearly proportional to temperature are available. (One such device is the LM34 available from National Semiconductor Corporation, Santa Clara, Calif.)

Physiologic sensors 103 output analog signals which may be digitized using an analog-to-digital (A/D) converter. It may be desirable to use an A/D converter in association with a microcontroller, that provides control functions for the A/D (such as sampling intervals, etc.), and, if necessary, serialization of the data to interface with a wireless networking device. Thus, in system 100, the output of physiologic sensors 103 are provided to a microcontroller 112 including an embedded A/D converter. (One such microcontroller that may be adapted for use in microcontroller 112 is the 16C77 device from Microchip Technology, Inc., Chandler, Ariz.) The output of microcontroller 112 is provided to a wireless networking device, in system 100 IEEE 802.15 device, commonly referred to as “Bluetooth.” For example bluetooth device 114 may be a Bluetooth-enabled wrist “watch, developed by the IBM Corporation, Armonk, N.Y. which has both infrared (IR) and Bluetooth wireless connectivity. In such an embodiment, link 115 may be an IR link between microcontroller 112 and Bluetooth device 114 in conjunction with an IrDA® protocol stack. (An IrDA® protocol stack may be provided by a Microchip MCP2120 infrared encoder/decoder used in conjunction with microcontroller 112. IrDA® is a set of protocols for two-way data communication over an infrared link promulgated by the Infrared Data Association, Walnut Creek, Calif.)

The Bluetooth specification provides for the secure transfer of data on the wireless link via encryption. In this way, sensitive personal information may be protected from compromise by “eavesdropping” by other Bluetooth enabled devices in the user's vicinity. Although the embodiment of stress monitoring system 100 illustrated in FIG. 1 refers to a Bluetooth wireless link, it would be appreciated by those of ordinary skill in the art that other wireless networking technologies may be used. For example, wireless networking in accordance with IEEE 802.11b, commonly referred to as “Wi-Fi” may also be used. Note that because of a shorter range, Bluetooth networking may be preferred however.

The physiologic sensor data is transmitted over the wireless network to a Bluetooth-enabled data processing system 116, which includes Bluetooth interface 118. In an embodiment using two or more sensors, microcontroller 112 may attach a header to the data to identify it to data processing system 116. Data processing system 116 may be a conventional personal computer or workstation or a personal digital assisted (PDA). Additionally, data processing system 116 may be embodied in a consumer electronic device such as a television, that includes (intelligence). That is, a consumer electronic device that has the capability of executing a software program. In particular, data processing system 116 includes monitor application 120, which is configured to receive the physiologic data from physiologic sensors 103. Additionally, as described further hereinbelow, monitor application 120 is configured to analyze the data with respect to the user's baseline and provide feedback to the user in accordance with the user's preselected profile. Feedback may be in the form of alerts displayed on a monitor or other display device 122. Additionally, audio responses, music, for example, may be provided in the form of analog audio output to headphones or speakers, or alternatively to an audio system (in either analog or digital form). These are generally embodied in audio system 124 in FIG. 1.

Although, as discussed above, the present inventive principles may be used in conjunction with different devices, having a stress level monitor deployed on a personal computer is particularly useful. Personal computers have become an essential tool in the modern workplace. While enhancing productivity, personal computers are also a source of frustration for many users. Most users have little or no exposure to technology, and a personal computer is, under the best of circumstances, a “black box” of which they have little or no understanding. Consequently, the personal computer environment itself is a significant source of stress for users confronting increasingly complex software which may also be subject to “crashes,” spam, viruses and similar tribulations. The present invention deployed on a personal computer provides an “integrated” countermeasure to such stress-producing events.

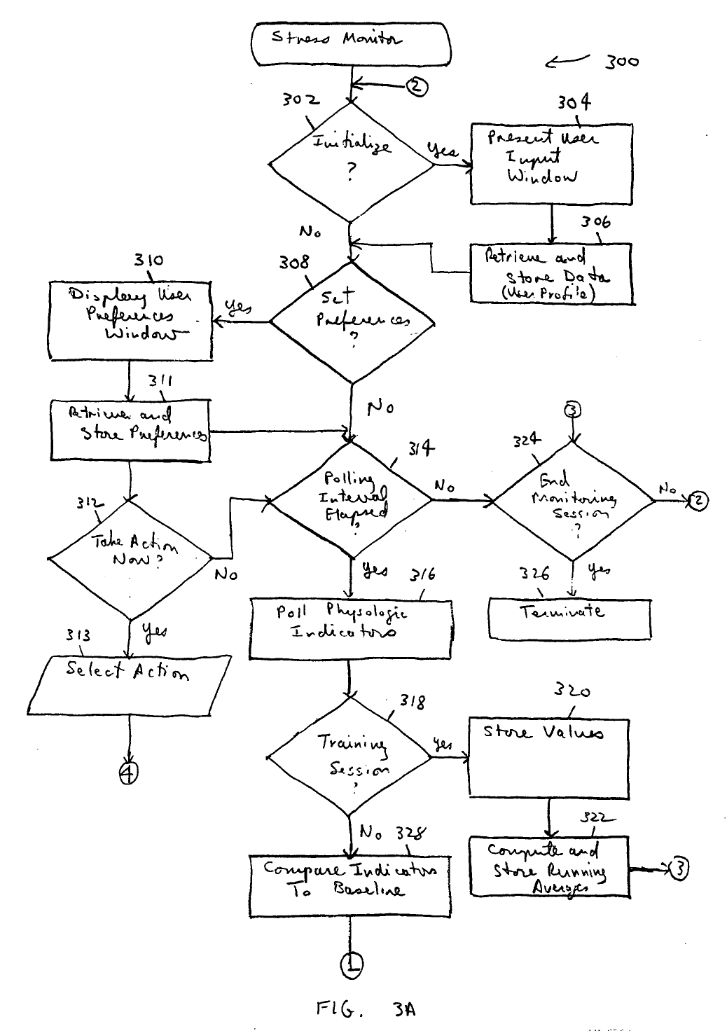

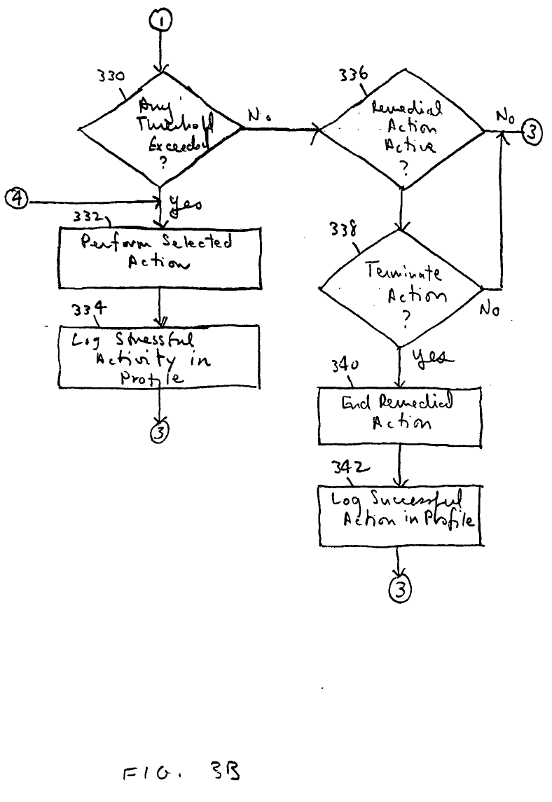

Refer now to FIG. 2 illustrating monitor application 120 in further detail. Application 120 may include input/output (I/O) logic which receives the physiologic data from Bluetooth interface 118, FIG. 1 as well as user input. I/O and control logic 202 may, for example, display graphical user interfaces (GUI) for receiving user input as discussed further below. Such GUIs are commonly referred to as dialog windows, dialog boxes or the like. As would be appreciated by person of ordinary skill in the art, such dialog windows may include fields for user entry of data as well as devices for selecting options, such as “radio” buttons or “check” boxes, for example. Input data provided by a user may include may include user profile data 204, which may be stored and used by analysis engine 206 to provide suggested remedies if the physiologic data indicate that the user is experiencing elevated levels of stress. Analysis engine 206 may make these determinations by comparing the physiologic data with baseline data 208. Baseline data 208 may include nominal data for physiologic indicators across a general population based on typical factors such as the user's weight, height, gender, etc. Alternatively, baseline data may be particularized to the user by analyzing the user's physiological data over a training session and establishing nominal values for the indicators for the particular user. These will be described further in conjunction with FIG. 3 hereinbelow.

Using the results from analysis engine 206, I/O and control logic 202 may provide output signals to a display, audio system or other such device to provide stress reducing suggestions and actions to the user.

Refer now to FIG. 3, illustrating, in flowchart form, stress monitor methodology 300 in accordance with the present inventive principles. Stress monitor methodology 300 may be used, for example, in conjunction with monitor application 120, FIGS. 1 and 2.

As previously noted, the principles of the present invention provide for the continuous monitoring of stress levels in individuals in an unobtrusive fashion, in the home or work environment. A user profile may be created by the user to individualize the levels associated with the physiologic indicators that trigger stress reducing responses by the monitoring methodology, as described below.

If a user profile is to be initialized, step 302, a user input window (a dialog window, dialog box, etc.) is presented in step 304. (A mechanism for presenting a user dialog box would be recognized as platform, i.e., operating system dependent, and moreover, it would be appreciated by those of ordinary skill in the art that the commonly employed operating systems on modern data processing systems, as well as PDA devices provide for high level operating system application program interfaces (API) for presenting such dialog windows.) In step 306, the user profile data is received and stored in the user profile, e.g., user profile 204, FIG. 2. User profile data may include, for example, the threshold levels, relative to a baseline or nominal value, at which selected stress-reducing responses are provided to the user. For example, a user profile may set the first threshold level (indicating, say mildly elevated stress, at a blood pressure of 110% of the baseline value for the user. A second threshold may be set at, for example, 125% of baseline, etc. In this way, a user may customize a profile of stress levels that indicate, for that user, successively increasing levels of stress. In particular, these levels may be set upon recommendation of the user's physician, or other healthcare professional, to accommodate a particular user sensitivity to stress or to mitigate preexisting medical conditions. Similar profiles may be established for other physiologic stress indicators, such as pulse rate and the like. Additionally, the profile may include a flag (which may be set by a “radio” button or similar device in the GUI) indicating that the user is in a stress-sensitive population, such as a pre-existing by pertensive condition or cardiovascular condition. This may be used to enable additional responses at the higher threshold levels. It would be appreciated by persons of ordinary skill in the art that these stress level thresholds are exemplary, and that the present inventive principles are not implicated by particular values for the thresholds. Referring again to decision block 302, if the user data is not to be initialized, steps 304 and 306 are bypassed via the “No” branch of decision block 302. User data may be initialized at the first launch of the stress monitoring application. Additionally, a user may choose to reset or modify his or her profile. This may be effected by, for example, providing a menu item in a GUI, the selection of which would cause decision block 302 to fall through its “yes” branch.

Decision block 308 permits a user to selectably set options, or preferences, to be used in conjunction with methodology 300. For example, a user may select the interval at which the physiologic data are polled. For example, a polling interval set by the user may override a default value, for example, ten minutes. Additionally, the preferences may be used to associate user selected stress relieving remedies with methodology 300. For example, a user may choose to associate a set of music files which a particular user finds relaxing with methodology 300. The user may also optionally select to override the stress-relief actions in the user profile. These override selections may be made the default alternatively, for the current session only. If, in step 308, the user has selected to set preferences, a user preferences dialog window is displayed in step 310 and in step 311 the preferences are retrieved and stored.

In step 312, it is determined if the user “manually” elects to initiate a stress-reducing action. In this way, a user anticipating that his or her current activity is likely to induce stress, may take action before symptoms of stress are detected. If, in step 312, the user elects to take a stress-reducing action, which may be effected by a menu selection or similar such device, the user selects the action in step 313, and process 300 proceeds to step 332 described hereinbelow. Step 313 may be effected by a dialog box or similar device for presenting and retrieving user options, however, any mechanism in the data processing arts for receiving user input may be used.

Returning to step 312, if the user does not elect to take a stress-relieving action, in step 314, a polling loop is entered. Decision block 314 in conjunction with decision blocks 302 and 308 form an event loop for methodology 300. Such event, loops would be recognized by persons of ordinary skill in the art as being associated with GUI-based applications.

In decision block 314, it is determined if the polling time interval has elapsed. As noted hereinabove, the polling interval may be set to a default value, for example, ten minutes, or maybe set a user preference. Until a polling interval elapses, process 300 loops over the decision blocks 314, 302 and 308. On the expiry of a polling interval, the physiologic indicators are polled. In other words, the values of the physiological stress indicators such as blood pressure, pulse rate, or skin resistance, or any combination thereof, are sampled from the data received by the wireless network interface, such as bluetooth interface 118, FIG. 1.

As previously described, a baseline for the physiologic stress indicators may be provided by typical values for a population based on characteristics such as age, height, weight, gender, etc. Alternatively, a baseline tailored to the particular user may be generated by “training” the monitoring application. The user may selectably train the monitoring process. In step 318, it is determined if the user has selected a training session. This may be effected by, for example, a menu item in a GUI-based system. If a training session has been selected, the polled physiologic indicators are stored, step 320. In step 322, running averages of the physiologic indicator data are generated and stored. Alternatively, limit the range of values of the indicator over the training session may be used in another embodiment of the present invention. The present inventive principles are not implicated by the particular measure of the baseline values of the indicators. Process 300 then proceeds to step 324 to determine if the user has selectably terminated the monitoring session (for example, by selecting an “Exit” or “Quit” option from a menu, user input options commonly used in GUI-based software applications). If so, in step 326 process 300 terminates. Otherwise, process 300 returns to step 302 and continues with its event loop.

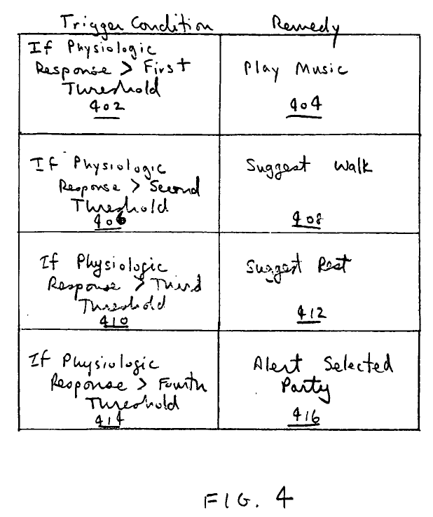

Returning to step 318, if the current session is not a training session, then process 300 determines if the user is experiencing elevated levels of stress as reflected in the values of the physiologic indicators polled in step 316. In step 328, the current values of the physiologic indicators are compared against the user's baseline. As previously discussed, the user profile may contain a set of thresholds for one or more physiologic indicators in which the thresholds, if exceeded, indicate the user may be experiencing elevated stress of levels. Additionally, larger deviations of the indicators from the baseline values may be correlated with increasing stress levels. Referring to FIG. 404, there is illustrated, in tabular form, a set of trigger conditions and an associated action or “remedy” which may be offered to the user. For example, if a physiologic response exceeds first threshold (402) then the users data processing system may automatically play some soothing music. As discussed hereinabove, the user may have preselected music which the particular user finds beneficial in such circumstances. The first threshold may be set in the user profile, for example, the threshold may be a blood pressure that is 110% of the baseline. If the physiologic response further deviates from the baseline and exceeds a second threshold (406), the user may be advised to break from their current activity and, take a walk, say (408). Again, the second threshold may be set in the user profile, for example, a blood pressure exceeding 125% of the baseline. A physiologic response exceeding a third threshold, higher than the second threshold, (410) may prompt the user with a more aggressive remedy, for example, suggesting the user rest (412). Even higher levels of stress as indicated by a physiologic response exceeding a fourth threshold (414) in the exemplary table of FIG. 4 may indicate a level of stress that might be potentially harmful to the user. This may be limited to a user population that is sensitive to excessive stress, such as a user population with a pre-existing cardiovascular condition which may be exacerbated by high levels of stress. In such a population, a physiologic indicator exceeding the fourth threshold in the Table of FIG. 4 may warrant alerting a selected third party (416). As discussed hereinabove, it may be advantageous to enable the user to selectively choose this option whereby those users in a population particularly sensitive to stress may select this alternative. It would be appreciated by those of ordinary skill in the art, that the foregoing examples are illustrative and that the present inventive principles are not implicated by particular selections of thresholds or remedies. Threshold conditions may constitute a threshold value for an individual indicator, or alternatively formed from a composite of indicator values. Thus, if any threshold in the user profile is exceeded, step 330, the action corresponding to the highest threshold exceeded is performed in step 332. In step 334, the stressful activity is logged to the user profile, and process 300 returns to step 324.

Returning to step 330, if a threshold is not exceeded, two possibilities exist. If a remedial action is active, the remedial action has reduced the user's stress, and the “Yes” branch in step 336 is taken, to optionally provide for the user to manually terminate the action via the “Yes” branch of step 338, and step 340. In step 342, the successful remedial action is logged in a user profile history, and process 300 returns to step 324. If the user chooses not to terminate the remedial action, process 300 proceeds via the “No” branch of step 338 to step 324. Note that in an alternative embodiment (not shown in FIG. 3B), the decision block 338 may be eliminated, and the remedial action terminated automatically.

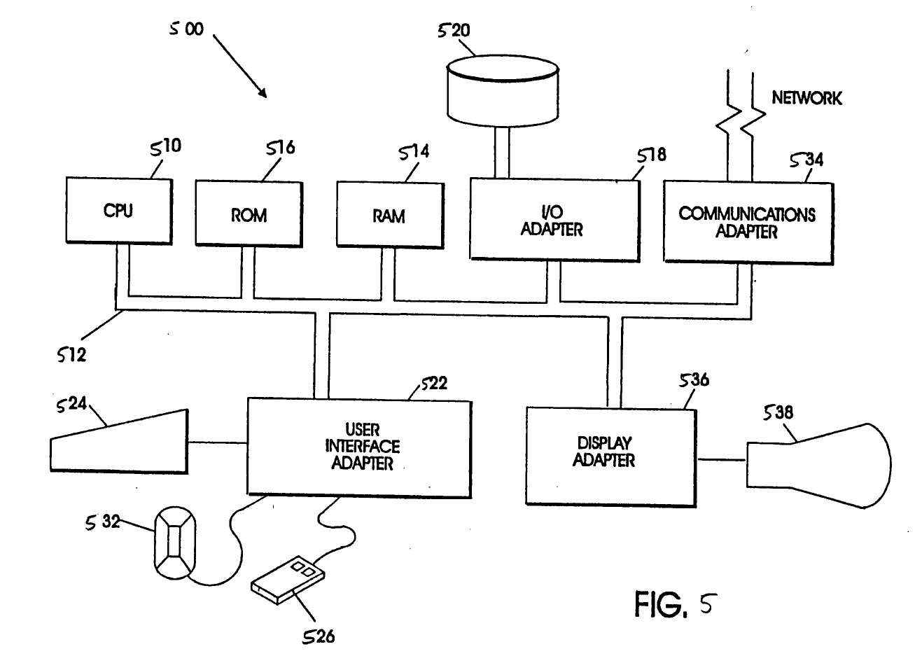

FIG. 5 illustrates an exemplary hardware configuration of data processing system 500 in accordance with the subject invention. The system in conjunction with the methodologies illustrated in FIGS. 3A-3B may be used to provide personal stress monitoring in accordance with the present inventive principles. Data processing system 500 includes central processing unit (CPU) 510, such as a conventional microprocessor, and a number of other units interconnected via system bus 512. Data processing system 500 also includes random access memory (RAM) 514, read only memory (ROM) 516 and input/output (I/O) adapter 518 for connecting peripheral devices such as disk units 520 to bus 512, user interface adapter 522 for connecting keyboard 524, mouse 526, trackball 532 and/or other user interface devices such as a touch screen device (not shown) to bus 512. User input received thereby may be passed to I/O and control logic 202, FIG. 2. System 500 also includes communication adapter 534 for connecting data processing system 500 to a data processing network, enabling the system to communicate with other systems, and display adapter 536 for connecting bus 512 to display device 538. CPU 510 may include other circuitry not shown herein, which will include circuitry commonly found within a microprocessor, e.g. execution units, bus interface units, arithmetic logic units, etc. CPU 510 may also reside on a single integrated circuit.

Preferred implementations of the invention include implementations as a computer system programmed to execute the method or methods described herein, and as a computer program product. According to the computer system implementation, sets of instructions for executing the method or methods are resident in the random access memory 514 of one or more computer systems configured generally as described above. These sets of instructions, in conjunction with system components that execute them may monitor a users stress related physiologic indicators and suggest remedial actions to the user as described hereinabove. Until required by the computer system, the set of instructions may be stored as a computer program product in another computer memory, for example, in disk drive 520 (which may include a removable memory such as an optical disk or floppy disk for eventual use in the disk drive 520). Further, the computer program product can also be stored at another computer and transmitted to the users work station by a network or by an external network such as the Internet. One skilled in the art would appreciate that the physical storage of the sets of instructions physically changes the medium upon which is the stored so that the medium carries computer readable information. The change may be electrical, magnetic, chemical, biological, or some other physical change. While it is convenient to describe the invention in terms of instructions, symbols, characters, or the like, the reader should remember that all of these in similar terms should be associated with the appropriate physical elements.

Note that the invention may describe terms such as comparing, validating, selecting, identifying, or other terms that could be associated with a human operator. However, for at least a number of the operations described herein which form part of at least one of the embodiments, no action by a human operator is desirable. The operations described are, in large part, machine operations processing electrical signals to generate other electrical signals.

Although the present invention and its advantages have been described in detail, it should be understood that various changes, substitutions and alterations can be made herein without departing from the spirit and scope of the invention as defined by the appended claims.

1. A method for personal stress monitoring comprising:
(a) receiving one or more physiologic indicators;
(b) comparing values of the one or more physiologic indicators to corresponding baseline values;
(c) determining if, in response to step (b) if the one or more physiologic indicators equals or exceeds at least one preselected threshold condition relative to baseline values; and
(d) if at least one threshold condition is equaled or exceeded in step (c), emitting a remedial action corresponding to a highest level threshold condition equaled or exceeded.

2. The method of claim 1 wherein the at least one threshold condition comprises user profile data.

3. The method of claim 1 wherein the at least one threshold condition comprises a condition relative to a single physiologic indicator value or a condition relative to a composite of physiologic indicator values.

4. The method of claim 1 further wherein the one or more physiologic indicators are received via a wireless network device from one or more sensors for sensing the user's corresponding physiologic indicator.

5. The method of claim 1 wherein a first set of baseline values are generated by training on a set of physiologic indicator values for the user.

6. The method of claim 5 wherein a second set of baseline values comprise a set of nominal values for a population based on one or more factors including height, weight and gender.

7. The method of claim 6 wherein the user profile includes values of the one or more factors.

8. A computer program product embodied in a computer readable medium for personal stress monitoring comprising programming instructions for:
(a) receiving one or more physiologic indicators;
(b) comparing values of the one or more physiologic indicators to corresponding baseline values;
(c) determining if, in response to step (b) if the one or more physiologic indicators equals or exceeds at least one preselected threshold condition relative to baseline values; and
(d) if at least one threshold condition is equaled or exceeded in step (c), emitting a remedial action corresponding to a highest level threshold condition equaled or exceeded.

9. The computer program product of claim 8 further comprising programming instructions for determining if a remedial action is manually selected; and retrieving a user selection for said remedial action.

10. The computer program product of claim 8 wherein the at least one threshold condition comprises a condition relative to a single physiologic indicator value or a condition relative to a composite of physiologic indicator values.

11. The computer program product of claim 8 further wherein the one or more physiologic indicators are received via a wireless network device from one or more sensors for sensing the user's corresponding physiologic indicator.

12. The computer program product of claim 8 wherein a first set of baseline values are generated by training on a set of physiologic indicator values for the user.

13. The computer program product of claim 12 wherein a second set of baseline values comprise a set of nominal values for a population based on one or more factors including height, weight and gender.

14. The computer program product of claim 8 wherein each threshold condition is associated with a remedial action, and wherein the programming instructions further include instructions for selectably overriding a remedial action.

15. A data processing system comprising:
(a) circuitry operable for receiving one or more physiologic indicators;
(b) circuitry operable for comparing values of the one or more physiologic indicators to corresponding baseline values;
(c) circuitry operable for determining if, in response to step (b) if the one or more physiologic indicators equals or exceeds at least one preselected threshold condition of a plurality of threshold conditions relative to baseline values; and
(d) circuitry operable for, if at least one threshold condition is equaled or exceeded in step (c), emitting a remedial action corresponding to a highest level threshold condition equaled or exceeded.

16. The data processing system of claim 15 wherein the at least one threshold condition comprises user profile data.

17. The data processing system of claim 15 wherein the at least one threshold condition comprises a condition relative to a single physiologic indicator value or a condition relative to a composite of physiologic indicator values.

18. The data processing system of claim 15 further wherein the one or more physiologic indicators are received via a wireless network device from one or more sensors for sensing the user's corresponding physiologic indicator.

19. The data processing system of claim 15 wherein a first set of baseline values are generated by training on a set of physiologic indicator values for the user.

20. The data processing system of claim 16 wherein user profile data further comprises one or more remedial actions associated with a corresponding one of the one or more threshold conditions, the data processing system further including circuitry operable for selectably overriding a remedial action in the user profile.
